(19) United States
(12) Patent Application Publication
(10) Pub. No.: US 2017/0231227 A1
(43) Pub. Date: Aug. 17, 2017

(54) ANTIFUNGAL-GRAFTED POLYOLEFIN

(71) Applicant: Joongmin Shin, Madison, WI (US)

(72) Inventor: Joongmin Shin, Madison, WI (US)

(21) Appl. No.: 15/435,916

(22) Filed: Feb. 17, 2017

Related U.S. Application Data
(60) Provisional application No. 62/296,147, filed on Feb. 17, 2016.

(51) Int. Cl. A01N 43/90; C08F 110/02; A23L 33/10; A61L 31/16; C08F 255/02; A01N 25/10; A61L 31/04

(52) U.S. Cl. CPC A01N 43/90; A01N 25/10; C08F 110/02; A61L 31/048; A61L 31/16; C08F 255/023; A23L 33/10; A61L 2300/404; A23V 2002/00

(57) ABSTRACT

Various embodiments disclosed relate to an antifungal-grafted polyolefin. The present invention provides an antifungal-grafted polyolefin comprising an antifungal-grafted repeating unit having the structure:

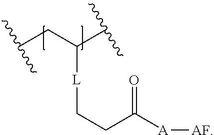

At each occurrence, -A- can be chosen from —O— and —NH—. At each occurrence, -AF can be an independently selected grafted antifungal compound. At each occurrence, -L- can be independently chosen from a bond and the structure:

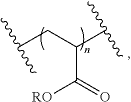

or a salt thereof.
At each occurrence —R can be independently chosen from —H and -AF. At each occurrence, the variable n is independently about 1 to about 100,000.

### CROSS-REFERENCE TO RELATED APPLICATION

This application claims the benefit of priority to U.S. Provisional Patent Application Ser. No. 62/296,147 filed Feb. 17, 2016, the disclosure of which is incorporated herein in its entirety by reference.

### BACKGROUND

Polymer surfaces are used to contact material sensitive to fungi in a variety of industries such as biomedical engineering, textiles, bioprocessing, and food processing. Most polymeric materials have a hydrophobic surface rather than hydrophilic. The hydrophobic surfaces are difficult to bond with polar materials, such as polar antifungal materials. Thus, physical deposition of antifungal compounds on the polymer surface usually results in a non-covalently bound coating that is readily removed from the polymer.

### SUMMARY OF THE INVENTION

In various embodiments, the present invention provides an antifungal-grafted polyolefin comprising an antifungal-grafted repeating unit having the structure:

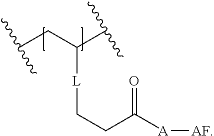

At each occurrence, -A- is chosen from —O— and —NH—. At each occurrence, -AF is an independently selected grafted antifungal compound. At each occurrence, -L- is independently chosen from a bond and the structure:

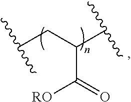

or a salt thereof.
At each occurrence —R is independently chosen from —H and -AF, and at each occurrence n is independently about 1 to about 100,000.

In various embodiments, the present invention provides an antifungal film including a polyolefin film including an antifungal-grafted repeating unit having the structure:

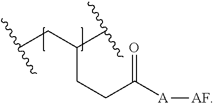

At each occurrence, -AF is a grafted natamycin (NA). The grafted antifungal has a concentration on the antifungal film of about 1 microgram/cm² to about 1000 microgram/cm².

In various embodiments, the present invention provides an antifungal-grafted polyolefin that can inhibit fungal growth via contact. In various embodiments, the antifungal-grafted polyolefin can be used as an effective food wrap or barrier for packaging materials such as melon, cheese, meats, and the like. In various embodiments, the antifungal-grafted polyolefin of the present invention can maintain its antifungal efficacy and can avoid migration of the antifungal compound into other materials such as food. By avoiding migration of the antifungal compound, the antifungal-grafted polyolefin can be safer and can earn regulatory approval more easily. In various embodiments, the antifungal-grafted polyolefin can be produced more quickly and with less expense than other antifungal-treated polymers. Unlike immersion or spraying of antifungal compounds for antifungal treatment, in various embodiments, antifungal treatments using the antifungal-grafted polyolefin can use a much smaller amount of antifungal for an equivalent antifungal effect.

### BRIEF DESCRIPTION OF THE FIGURES

The drawings illustrate generally, by way of example, but not by way of limitation, various embodiments discussed in the present document.

FIG. 1 illustrates a schematic representation of a process of UV introduced acrylic acid (AA) and natamycin grafting, in accordance with various embodiments.

FIG. 2 illustrates a non-oxygen pouch system and UV treatment for surface grafting process, in accordance with various embodiments.

FIG. 3 illustrates grafting percentage (%) AA onto low density polyethylene (LDPE) films in different solvent solutions, in accordance with various embodiments.

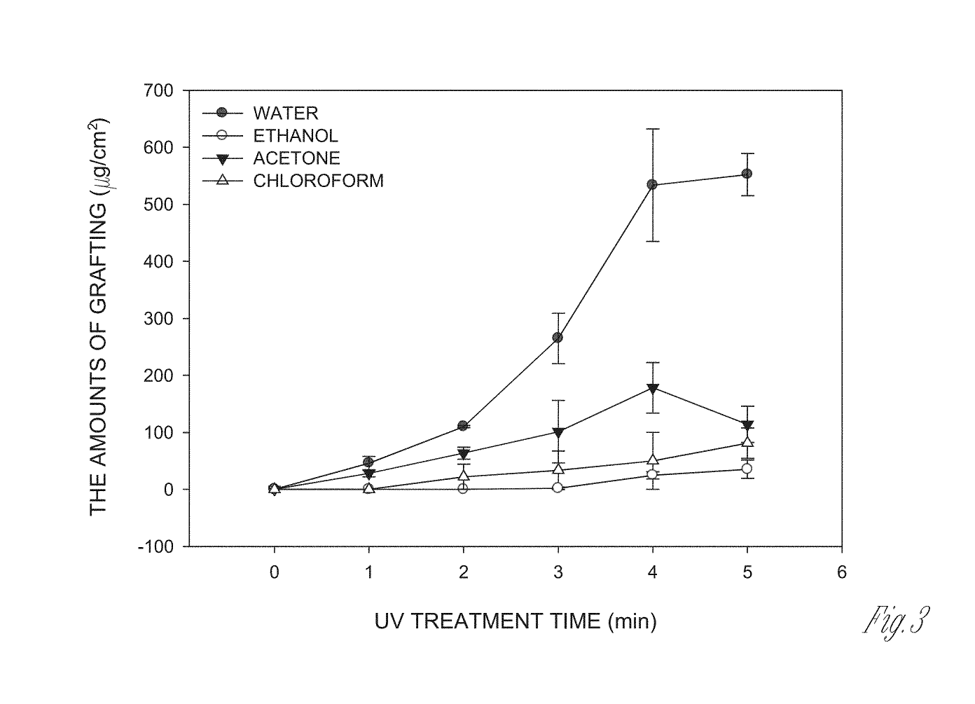

FIG. 4 illustrates grafting percentage (%) AA onto LDPE films in different concentrations of water/acetone solutions, in accordance with various embodiments.

FIG. 5 illustrates a migration assay on agar with 100 μl of Penicillium. chrysogenum inoculum, in accordance with various embodiments.

FIGS. 6A-B illustrate mechanical properties of the LDPE films with Natamycin under different UV treatments, with FIG. 6A illustrating tensile strength (TS) and FIG. 6B illustrating elongation at break (% E), in accordance with various embodiments.

FIG. 7 illustrates ATR-FTIR spectra of LDPE grafted with NA under different UV treatment times, in accordance with various embodiments.

FIGS. 8A-B illustrate populations of Saccharomyces cerevisiae inoculated on DRBC media and overlaid with active antimicrobial film, with the area covered by the film enumerated for populations of the yeast and mold, with 8A illustrating P. chrysogenum and 8B illustrating S. cerevisiae, in accordance with various embodiments.

FIGS. 9A-B illustrate suppression of P. chrysogenum and S. cerevisiae by UV-natamycin films on fresh cut cantaloupes, with FIG. 9A illustrating P. chrysogenum and FIG. 9B illustrating S. cerevisiae, in accordance with various embodiments.

FIGS. 10A-E illustrate SEM images of the Natamycin coated films under different UV treatment, in accordance with various embodiments.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 600%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “heterocyclyl” as used herein refers to aromatic and non-aromatic ring compounds containing three or more ring members, of which one or more is a heteroatom such as, but not limited to, N, O, and S.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “number-average molecular weight” (Mₙ) as used herein refers to the ordinary arithmetic mean of the molecular weight of individual molecules in a sample. It is defined as the total weight of all molecules in a sample divided by the total number of molecules in the sample. Experimentally, Mₙ is determined by analyzing a sample divided into molecular weight fractions of species i having nᵢ molecules of molecular weight Mᵢ through the formula Mₙ=ΣMᵢnᵢ/Σnᵢ. The Mₙ can be measured by a variety of well-known methods including gel permeation chromatography, spectroscopic end group analysis, and osmometry. If unspecified, molecular weights of polymers given herein are number-average molecular weights.

The term “weight-average molecular weight” as used herein refers to M_{w}, which is equal to ΣMᵢ²nᵢ/ΣMᵢnᵢ, where nᵢ is the number of molecules of molecular weight Mᵢ. In various examples, the weight-average molecular weight can be determined using light scattering, small angle neutron scattering, X-ray scattering, and sedimentation velocity.

The term “solvent” as used herein refers to a liquid that can dissolve a solid, liquid, or gas. Non-limiting examples of solvents are silicones, organic compounds, water, alcohols, ionic liquids, and supercritical fluids.

The term “room temperature” as used herein refers to a temperature of about 15° C. to 28° C.

Herein, when it is designated that a variable in the structure can be “a bond,” the variable can represent a direct bond between the two groups shown as linked to that variable, such as a single bond.

As used herein, the term “polymer” refers to a molecule having at least one repeating unit and can include copolymers.

The term “mil” as used herein refers to a thousandth of an inch, such that 1 mil=0.001 inch.

In various embodiments, salts having a positively charged counterion can include any suitable positively charged counterion. For example, the counterion can be ammonium(NH₄⁺), or an alkali metal such as sodium (Na⁺), potassium (K⁺), or lithium (Li⁺). In some embodiments, the counterion can have a positive charge greater than +1, which can in some embodiments complex to multiple ionized groups, such as Zn²⁺, Al³⁺, or alkaline earth metals such as Ca²⁺ or Mg²⁺.

In various embodiments, salts having a negatively charged counterion can include any suitable negatively charged counterion. For example, the counterion can be a halide, such as fluoride, chloride, iodide, or bromide. In other examples, the counterion can be nitrate, hydrogen sulfate, dihydrogen phosphate, bicarbonate, nitrite, perchlorate, iodate, chlorate, bromate, chlorite, hypochlorite, hypobromite, cyanide, amide, cyanate, hydroxide, permanganate. The counterion can be a conjugate base of any carboxylic acid, such as acetate or formate. In some embodiments, a counterion can have a negative charge greater than −1, which can in some embodiments complex to multiple ionized groups, such as oxide, sulfide, nitride, arsenate, phosphate, arsenite, hydrogen phosphate, sulfate, thiosulfate, sulfite, carbonate, chromate, dichromate, peroxide, or oxalate.

The polymers described herein can terminate in any suitable way. In some embodiments, the polymers can terminate with an end group that is independently chosen from a suitable polymerization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

### Antifungal-Grafted Polyolefin.

In various embodiments, the present invention provides an antifungal-grafted polyolefin including an antifungal-grafted repeating unit having the structure:

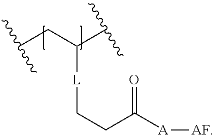

At each occurrence, -A- can be chosen from —O— and —NH—. At each occurrence, -AF can be an independently selected grafted antifungal compound. At each occurrence, -L- can be independently chosen from a bond and the structure:

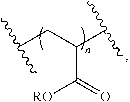

or a salt thereof.
At each occurrence —R can be independently chosen from —H and -AF. At each occurrence n is independently about 1 to about 100,000.

At each occurrence, -A- can be chosen from —O— and —NH—. The variable -A- can be —O—, and -AF can be an antifungal compound grafted to the polyolefin via an esterification reaction of an —OH group on an ungrafted antifungal compound and a —C(O)OH group on the polyolefin. The variable -A- can be —NH—, and -AF can be an antifungal compound grafted to the polyolefin via an amidization reaction of an —NH₂ group on an ungrafted antifungal compound and a —C(O)OH group on the polyolefin.

At each occurrence, -AF can be an independently selected grafted antifungal compound. The antifungal compound can be any suitable antifungal compound that includes an —OH or —NH₂ group that can react with a —C(O)—OH group to form a —C(O)—O— bond (esterification) or a C(O)—NH— bond (amidization). The grafted antifungal compound can be grafted amphotericin B, candicidin, filipin III, hamycin, natamycin, nystatin, rimocidin, efinaconazole, fluconazole, isavuconazole, posaconazole, ravuconazole, voriconazole, anidulafungin, ciclopirox, flucytosine, or grafted undecylenic acid. The grafted antifungal compound can be grafted natamycin. Natamycin can be grafted via any suitable —OH or —NH₂ group thereon and has the structure:

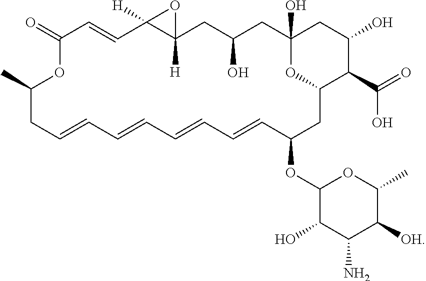 [SMILES: C[C@@H]1C/C=C/C=C/C=C/C=C/[C@H](OC2O[C@H](C)[C@@H](O)[C@H](N)[C@@H]2O)C[C@@H]2O[C@](O)(C[C@@H](O)C[C@H]3O[C@@H]3/C=C/C(=O)O1)C[C@H](O)[C@H]2C(=O)O]

At each occurrence, -L- can be independently chosen from a bond and the structure:

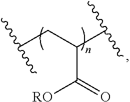

or a salt thereof.
At each occurrence —R can be independently chosen from —H and -AF. At each occurrence n can be independently about 1 to about 100,000, or about 1 to about 1,000, or about 1 to about 10, or about 1, or less than, equal to, or greater than about 2, 3, 4, 5, 6, 7, 8, 9, 10, 12, 14, 16, 18, 20, 25, 30, 35, 40, 45, 50, 60, 70, 80, 90, 100, 150, 200, 250, 500, 750, 1,000, 1,500, 2,000, 2,500, 5,000, 10,000, 15,000, 20,000, 25,000, 50,000, or about 100,000 or more.

The polyolefin can be a homopolymer or a copolymer. The polyolefin can be branched or linear. The polyolefin can be ultra high molecular weight polyethylene (UHMWPE), ultra low molecular weight polyethylene (ULMWPE), high molecular weight polyethylene (HMWPE), high density polyethylene (HDPE), high density cross-linked polyethylene (HDXLPE), cross-linked polyethylene (PEX or XLPE), medium density polyethylene (MDPE), low density polyethylene (LDPE), linear low density polyethylene (LLDPE) and very low density polyethylene (VLDPE). The polymer can be LDPE.

The polyolefin can be a polymer of one or more compounds that are each independently a substituted or unsubstituted (C₂-C₂₀)hydrocarbon including at least one carbon-carbon unsaturated nonaromatic bond. The polyolefin can further include another repeating unit, the other repeating unit in a block or random arrangement in the polyolefin with the antifungal-grafted repeating unit, the other repeating unit being free of the grafted antifungal compound and having the structure:

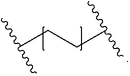

A molar ratio of the antifungal-grafted repeating unit to the other repeating unit, or to all repeating units not including a grafted antifungal, can be about 0.001:99.999 to about 99.999:0.001, or about 0.1:99.9 to about 99.9:0.1, or about 0.001:99.999 or less, or less than, equal to, or greater than about 0.01:99.99, 0.1:99.9, 1:99, 2:98, 3:97, 4:96, 5:95, 6:94, 8:92, 10:90, 15:85, 20:80, 30:70, 40:60, 50:50, 60:40, 70:30, 80:20, 85:15, 90:10, 92:8, 94:6, 95:5, 96:4, 97:3, 98:2, 99:1, 99.9:0.1, 99.99:0.01, or about 99.999:0.001 or more.

A film can include the polyolefin. The film can be any suitable film. The film can be food wrap film. The film can have any suitable thickness, such as about 0.01 microns to about 1 mm, about 1 micron to about 30 microns, or about 0.01 microns or less, or less than, equal to, or greater than about 0.1 microns, 1, 2, 3, 4, 5, 6, 7, 8, 9, 10, 11, 12, 13, 14, 15, 16, 17, 18, 19, 20, 21, 22, 23, 24, 25, 26, 28, 30, 35, 40, 45, 50, 55, 60, 70, 80, 90, 100, 125, 150, 175, 200, 250, 500, 750, or about 1 mm or more. Any suitable wt % of the film can be the antifungal-grafted polyolefin, such as about 1 wt % to about 100 wt % of the film, about 80 wt % to about 100 wt %, or about 1 wt % or less, or less than, equal to, or greater than about 2 wt %, 3, 4, 5, 6, 8, 10, 12, 14, 16, 18, 20, 25, 30, 35, 40, 45, 50, 55, 60, 65, 70, 75, 80, 85, 86, 88, 90, 91, 92, 93, 94, 95, 96, 97, 98, 99, 99.9, 99.99, or about 99.999 wt % or more.

The grafted antifungal can have any suitable concentration on the polyolefin (e.g., on the surface of the polyolefin, such as on the surface of a film including the polyolefin), such as about 0.001 microgram/cm² to about 100,000 microgram/cm², about 1 microgram/cm² to about 1000 microgram/cm², or about 0.001 microgram/cm² or less, or less than, equal to, or greater than about 0.01 microgram/cm², 0.1, 1, 2, 3, 4, 5, 10, 15, 20, 25, 30, 40, 50, 75, 100, 125, 150, 175, 200, 250, 500, 750, 1,000, 1,500, 2,000, 2,500, 5,000, 10,000, 15,000, 20,000, 25,000, 50,000, 75,000, or about 100,000 microgram/cm² or more.

### Method of Using the Antifungal-Grafted Polyolefin.

In various embodiments, the present invention provides a method of using the antifungal-grafted polyolefin. The antifungal-grafted polyolefin can be used in any suitable way. The method can include placing the antifungal-grafted polyolefin in contact with a food item or a living human or animal body, such as via contacting the food item with a food wrap including the antifungal-grafted polyolefin to prevent or reduce fungal growth on the food item, or such as via contacting the living human or animal body with a medical implant to prevent or reduce fungal growth on the medical implant in the body.

### Medical Device or Medical Implement.

In various embodiments, the present invention provides a medical device including the antifungal-grafted polyolefin. The medical device can be any suitable medical device that is designed to be contacted with a living human or animal body. The medical device can be a catheter or an orthopedic device. The medical implement can be any suitable medical implement designed to be used in connection with medical treatment or medical research, such as a cleanroom device, a bag, a door kick plate, intervention equipment, an incubator, or a combination thereof.

### Packaged Food Item.

In various embodiments, the present invention provides a packaged food item including the antifungal-grafted polyolefin. The food item can be any suitable food item. The antifungal-grafted polyolefin can form part of the packaging that contacts the food item and can prevent or reduce fungal growth on the food item. The antifungal-grafted polyolefin can be part of a food wrap on the item.

### Method of Forming the Antifungal-Grafted Polyolefin.

In various embodiments, the present invention provides a method of forming the antifungal-grafted polyolefin. The method can be any suitable method that can form an embodiment of the antifungal-grafted polyolefin described herein. The method can include contacting a polyolefin with a free radical initiator and acrylic acid or a salt there of to form an acrylic acid-grafted polyolefin. The method can include contacting the acrylic-acid-grafted polyolefin with an antifungal compound, to form the antifungal-grafted polyolefin. The method can further include exposing the polyolefin to light, which can be provided by any suitable light source such as an ultra violet light source, a light emitting diode, or ambient light.

### Examples

Various embodiments of the present invention can be better understood by reference to the following Examples which are offered by way of illustration. The present invention is not limited to the Examples given herein.

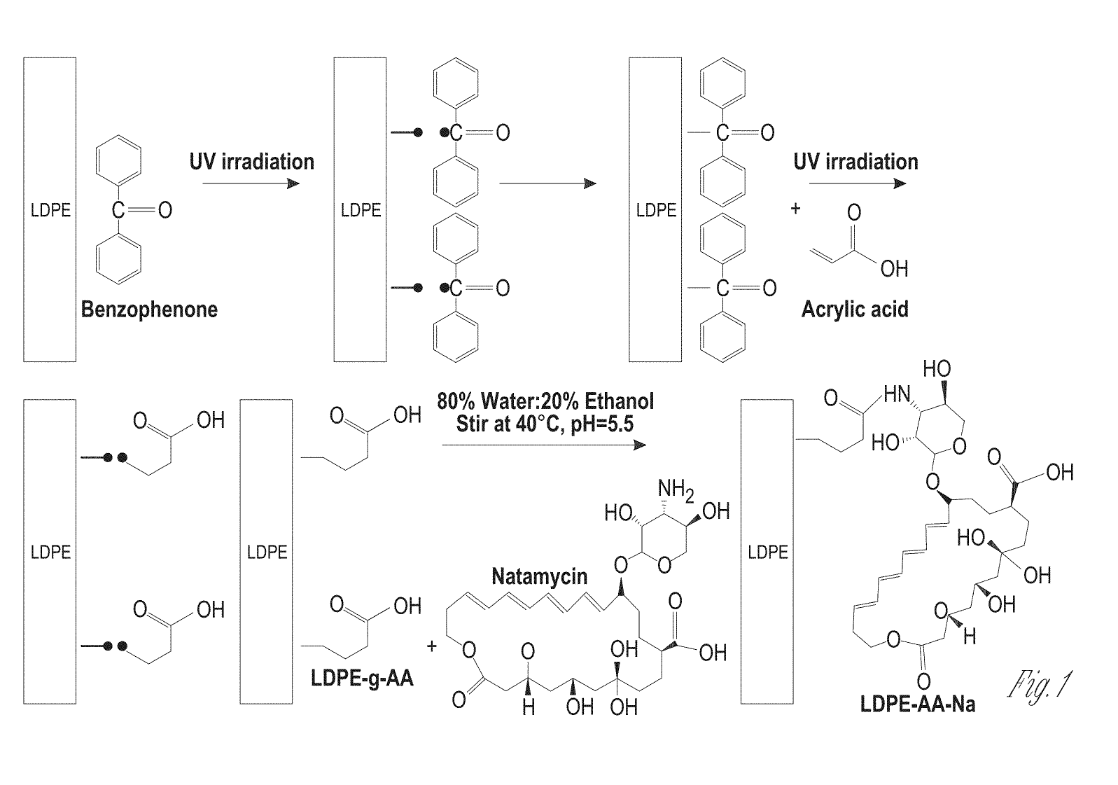

According to some embodiments of the present invention, an active non-migratory antifungal LDPE polymer for use in food packaged applications was developed. Functional acrylic acid monomer was grafted on the LDPE film surface by photo-initiated graft polymerization using Ultra Violet light irradiation (from 0 to 5 min). Natamycin, an antifungal agent, was applied to the treated film to bind with the pendent functional groups and its performance was evaluated against mold and yeast. The procedure schematics for the photoinitiated graft polymerization is shown in FIG. 1. The grafted amounts were determined by gravimetric measurement and dye absorbance. Attenuated Total Reflectance/Fourier Transfer Infrared Spectroscopy, scanning electron microscopy, and mechanical strength test was used to characterize film properties. The antifungal efficacy of the film was evaluated with Saccharomyces cerevisiae and Penicillium chrysogenum on growth media and fresh cut cantaloupe. The grafted group yield increased with the ultraviolet exposure time, the graft polymerization yielded up to 49.87 μg/cm². The natamycin grafted films inhibited mycelium formation of P. chrysogenum spores by over 60%. However, due to the thickness of film (less than 0.5 mil), the longer UV exposure reduced, the more mechanical strength. The application of such non-migratory active packaging film represents a promising approach to maintaining food quality with reduced additive.

### Example 1. Materials and Methods

### Example 1.1. Materials

Natamycin was donated from Danisco (Fayetteville, N.C., USA). Acrylic acid (AA) and Acid Orange 8 dye (AO8) were purchased from Acros Organics (Fair Lawn, N.J.). Monobasic sodium phosphate and Disodium phosphate, Benzophenone (BP), acetone, chloroform, ethanol, and hexane were purchased from Fisher Scientific (Waltham, Mass.). LDPE cling wrap (Saran, SC Johnson, Milwaukee) was purchased from the local grocery store. Tween 80 (polyoxyethylene sorbitan monooleate) was purchased from VWR (Chester, Pa.). Dichloran Rose Bengal Chloramphenicol (DRBC) Agar, and Potato Dextrose Agar (PDA) were purchased from Difco Laboratories (Detroit, Mich.). Dry yeast (Saccharomyces cerevisiae, Fleischmannes, Cincinnati, Ohio) and fresh cantaloupes were purchased from local grocery store.

### Example 1.2. Preparation of LDPE Film

LDPE clings wrap films with an average thickness of 12.5 μm were cut into a circle piece which has 100 mm diameter. The film was then cleaned with ethanol and acetone, and rinsed in deionized water sequentially (30 min per repetition per solvent), in order to remove a number of additives such as antioxidants and UV stabilizers which may interfere with the graft polymerization process and subsequent antioxidant activity assays. Then, the sample was dried under fume hood for 24 hours at ambient temperature.

### Example 1.3. Preparation of BP Coated LDPE Film

The LDPE cling wrap was dipped in various solutions, including acetone, ethanol, chloroform, and hexane containing 0.2M BP for 12 hr and dried through ventilation hood for 30 min to evaporate the solvent. The amount of BP absorbed by the film was determined gravimetrically using microbalance. The estimated total weight gain was calculated as follows:

$$Total Weight Gain ( TG ) \% = \frac{Wg - Wo}{Wo} \times 100 ( \% ) ,$$

where Wg and Wo are the weights of the film sample after and before dipping in solvents, respectively.

### Example 1.4. UV-Treated and Graft Polymerization

The LDPE film samples were dipped in Chloroform solution containing 0.2M Benzophenone (BP) for 12 hours, and dried through ventilation hood for 30 min to evaporate the solvent. Then, 1 piece of film sample was placed in an open Pyrex petri dish (100 mm×15 mm, Corning, Midland, Mich.) with 10 ml of 20% acrylic acid (AA) solution.

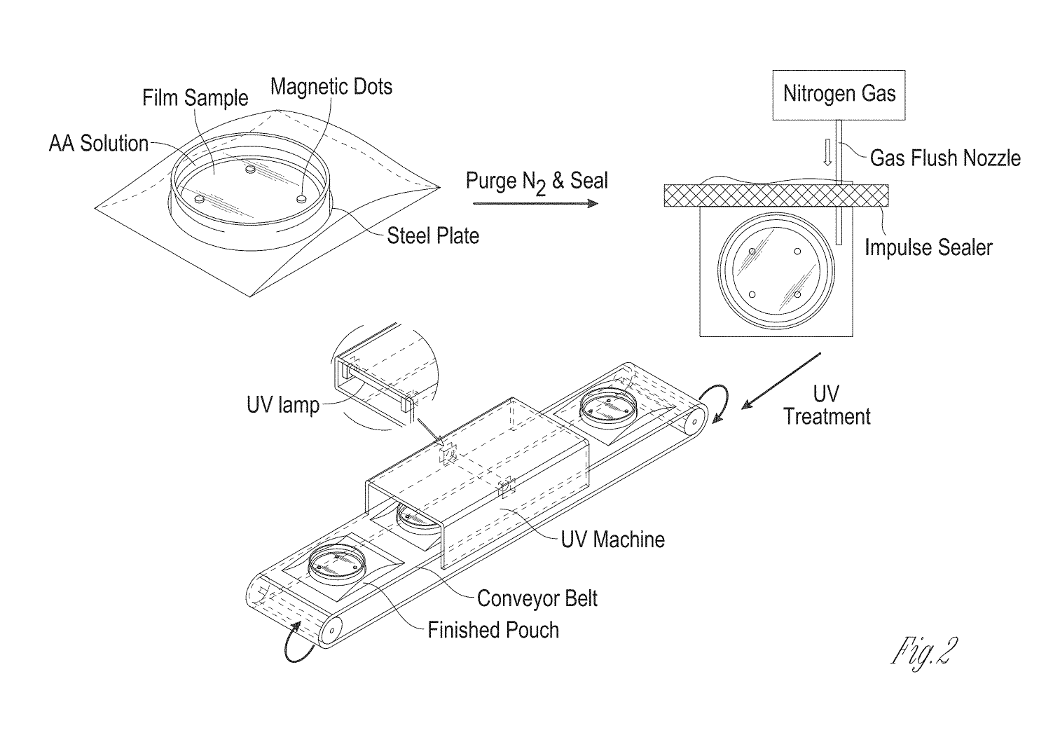

Under the petri dish, a metal disk (100 mm×1 mm) was attached using adhesive, and 4 magnetic dots were placed to fix the film on the dish. Four different solvents, including acetone, ethanol, chloroform, and acetone were used to make the 20% AA solution. Because oxygen could scavenge free-radicals, which are critical for the graft polymerization on LDPE, an inert atmosphere without oxygen was prepared and maintained by gas flush in a transparent high barrier film pouch with over 98% UV transmittance (Lid 1050, Cyovac, Elmwood Park, N.J.). First of all, the AA contained petri dish was contained in 200 mm×100 mm of high barrier pouches. Then, the pouches were flushed with 100% N₂, compensatorily vacuumed (to minimize the volume of the pouch), and sealed hermetically using a gas flush/vacuum dual mode impulse sealer (Model 14-TT-VAC-1/4, Therm-O-Seal Co., Mansfield, Tex.). The sealed pouches, were then exposed to 27.9 watts/cm² (180 watts/in²) ultraviolet (UV) light at a distance of 2 cm using UV-100 UV curing equipment (Innovative Machines, Jenison, Mich.). Iron metal halide lamp that generated UV emission from 250 to 320 mm was used for the grafting. The designed pouches used for grafted polymerization are shown in FIG. 2. The thin (12.5 μm) LDPE clip wrap film was easily damaged by the emitted heat from UV lamp. To avoid the film's thermal degradation, the speed of UV machine's conveyor belt was set to have the 20 sec treatment cycle. After 20 sec treatment, all films were cooled down for 10 sec, and the treatment cycle was repeated until the accumulated UV exposure time on the film reached a desirable level. The applied time of UV exposure was from 0 to 5 min at room temperature (25° C.). All treated films were rinsed with distilled water twice at 50° C. to remove unreacted AA monomer. Finally, LDPE-AA films were dipped in solution (80% water/20% ethanol) containing 1% natamycin and 0.05M of sodium phosphate buffer (5.0 pH) for 12 hours, at 40° C. After the incubation, all films were rinsed with distilled water three times at 40° C., dried, and stored in a dark desiccator until they were used.

### Example 1.5. The Efficiency of AA Grafting

The efficiency of AA grafting was expressed as the weight increase per surface area of the sample. After the grafting reaction were carried out, the films were washed and dried, as is described above, and the total weight was measured using microbalance. The results were calculated as follows:

$$Total amounts of grafting ( μ g \text{/} cm ) = \frac{Wp - Ws}{Sa}$$

where Wp is the weights of the grafted products, Ws is the weights of substrates, Sa is surface area of film.

### Example 1.6. Concentration of the Grafted Natamycin

Natamycin on the sample's surface was quantified using acid orange (AO) 8 dye assays. All samples were cut to 25.4×25.4 mm square, 5 films were immersed in 20 ml aqueous AO 8 dye solution (2.2×10⁻⁵ M) in amber vials (40 ml). The vials were stored at ambient temperature for 1 hour to give enough dye absorption on films. Then, the dye absorption in the dye solution was measured before and after contact with the films with Agilent 8453 UV-VIS spectrometer (Agilent Technologies, Santa Clara, Calif.) at 584 nm. Natamycin concentrations in the AO8 solution were determined from a standard curve, and the natamycin contents on the surface of the film were calculated based on such concentration and the sample dimensions. Three replicates per treatment were performed for this assay.

### Example 1.7. Characterization of Films Using ATR-FTIR

Changes in surface chemistry from the modified PE films were evaluated with a Nicolet 5 SXC FTIR system with an ATR accessory (Thermo Scientific, Madison, Wis.). For a total of three replicates, absorbance was measured in three different spots per film, and three films were analyzed per treatment. The ATR-FTIR spectra were performed in the absorption mode with a resolution of 4 cm⁻¹ and 42 scans.

### Example 1.8. Inoculum Preparation of Penicillium chrysogenum and Saccharomyces cerevisiae

Preparation of P. chrysogenum ATCC 10106 was accomplished as follows. Pure cultures of the P. chrysogenum strain were maintained on Potato Dextrose Agar (PDA, Difco, Detroit, Mich.). Inoculum was produced by growing the organism on the media slants for 8 days at 25° C., at the end of which the entire slant surface was covered with spores of the mold. 5.0 ml of sterile 0.01% Tween 80 (VWR, Chester, Pa.) was then added to each of the slants and the tubes were shaken gently to disperse the spores. The number of spores was determined using a Bright-Line™ hemocytometer (Hausser Scientific, Horsham, Pa.). The spore suspension was also serially diluted and spread on PDA medium to confirm the spore concentrations. The stock spore suspension of P. chrysogenum contained approximately 108 CFU/ml. All reagents and equipment were sterilized by autoclaving at 121° C. for 45 min. Dry yeast (Saccharomyces cerevisiae, Fleischmanns Inc., Cincinnati, Ohio) was obtained at a local grocer. The yeast culture was prepared based on package instructions. 1 g of the yeast was added to 10 mL of sterile water kept at 37.8° C. This stock inoculum of the yeast contained approximately 108 CFU/ml.

### Example 1.9. Evaluation of the Antifungal Activity Against S. cerevisiae and P. chrysogenum

The S. cerevisiae stock inoculum was serially diluted by transferring a 1 ml aliquot of the stock inoculum to 9 ml BPW. Subsequent dilutions were achieved by transferring 1 ml of diluted sample into 9 ml BPW. 0.1 ml aliquots of the serially diluted samples were spread plated onto Dichloran Rose Bengal Chloramphenicol (DRBC) Agar. Then, 103 mm diameter of round film cut was overlaid on the inoculated DRBC media to determine film efficacy. The plates were incubated at 25° C. for 5 days and enumerated to determine population of S. cerevisiae.

A 100 μl (approximately 976 spores) suspension of P. chrysogenum was spread plated onto DRBC Agar. Following this, 103 mm diameter of round film cut were overlaid on the inoculated DRBC media to determine film efficacy. The plates were incubated at 25° C. for 5 days, and the number of spore forming units (SPU) of P. chrysogenum were periodically enumerated to determine the efficacy of the films against the mold. In order to determine if the grafted agents are migrated from the treated film to agar, 4 min UV treated Natamycin films were cut into 38 mm discs and placed onto a nutrient agar with the 100 μl mold suspension (P. chrysogenum) which is described above.

### Example 1.10. Evaluation of the Antifungal Activity S. cerevisiae and P. chrysogenum on Fresh-Cut Cantaloupe

Studies were conducted to test the film efficacy on food products. Cantaloupes (Cucumis melo L.) without any visual defects were purchased from a wholesale market and stored at 3° C. for 1 day before the experiment. Cantaloupes were washed with 200 ppm (200 μL/L water) of sodium hypochlorite solution (pH 6.5) for 5 min. The sanitized cantaloupes were peeled, halved, and cut into 50×50×8 mm blocks. Then, the blocks were rinsed with 100 ppm (100 μL/L water) of sodium hypochlorite solution for 2 min and drained for 30 min. All utensils (knives, cutting boards, and other equipment which come into contact with the fruits) were sanitized by immersion in 1000 ppm (1000 μL/L water) of sodium hypochlorite solution for 1 hr before cutting. Each 0.1 ml of S. cerevisiae and P. chrysogenum were inoculated with on the surface of each cantaloupe separately. A disposable spreader was used to spread the inoculum on all sides of the food product using sterile forceps to hold the food sample. The sample was allowed to air dry for 10 minutes in the laminar hood and was then overwrapped with the film treatments. Inoculated cantaloupes which were overwrapped with untreated LDPE were used as controls. All samples were then incubated at 3° C. to monitor the efficacy of the films against the germination/growth of the inoculated species.

### Example 1.11. Film Surface Morphology Analysis

The surface morphology of the UV treated films was tested by employing a scanning electron microscope (Quanta 250, FEI Co. Ltd., Hillsboro, Oreg.) and compared to control film (0 min treated LDPE). All samples were cut with a sharp scalpel and were mounted on aluminum stubs using carbon adhesive tape and sputter coated with platinum (Pt) on the surfaces and fractured cross-sections of films. The samples were examined using an accelerating voltage of 10 kV.

### Example 1.12. Statistical Analysis

Statistical evaluation of the data was performed using SPSS ver. 2015 (SPSS Inc. Chicago, Ill.). One-way analysis of variance (ANOVA) followed by Tukey's honestly significant difference (HSD) multiple comparison was conducted to determine the difference. Significance levels were reported at the 95% confidence level (p<0.05).

### Example 2. Results and Discussion

### Example 2.1. The Efficiency of BP Coating Solvents

Bezophenone is one of the commonly used photo-initiators. Under UV treatment, BP absorbs energy and is excited to singlet state, and releases to more stable triplet state. Under the excited state, BP abstracts hydrogen from the chains of LDPE and generates free radicals on LDPE. Thus, BP serves a critical role in the grafting process. In order to find a suitable solvent which can penetrate the LDPE film rapidly and carry the BP molecule on the surface of the films, various reagents were applied as solvents of BP, including n-Hexane, acetone, chloroform, and ethanol, as is shown in Table 1. The highest BP absorption on LDPE was observed with chloroform. Thus, Chloroform was used for all subsequent film process.

TABLE 1
Chloroform coated the highest amount of BP on the film. Percent
gain is given as mean ± SD of triplicate measurements.
For each quality parameter, means with different superscripts
within a column indicate significant differences (P < 0.05).

| Solvents | % gain |
|---|---|
| Chloroform | 1.30 ± 0.05^{a} |
| Ethanol | 0.30 ± 0.10^{c} |
| Acetone | 0.66 ± 0.08^{b} |
| Hexene | 0.67 ± 0.12^{b} |

### Example 2.2. The Efficiency of AA Grafting

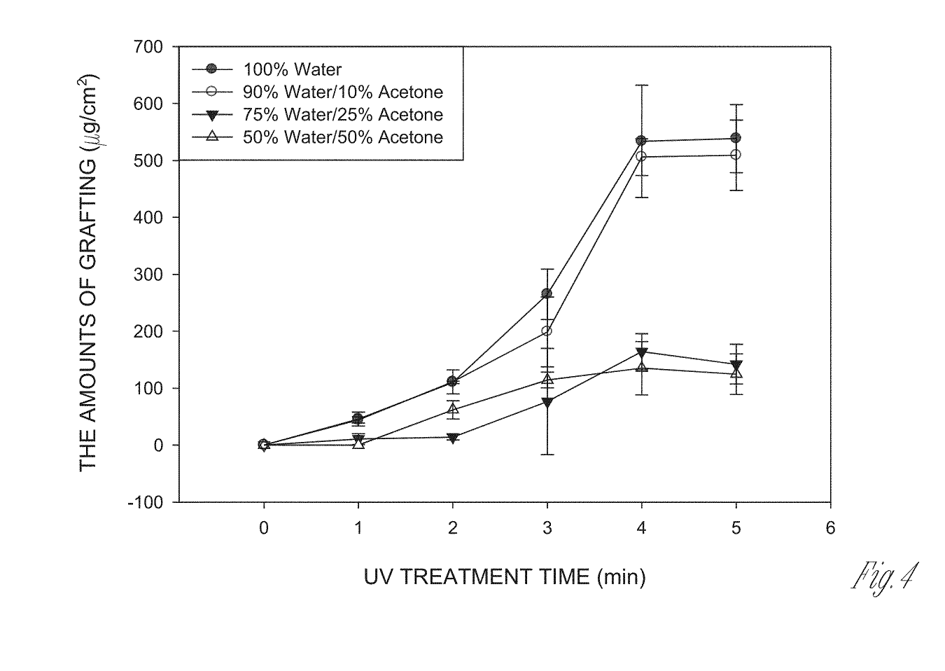

To provide for more conditions for bonding and The efficiency of AA grafting measured for control and treated LDPE films to determine the effectiveness of solvents (water, ethanol, chloroform, and acetone) on AA grafting of the films. Solvent plays an important role in affecting initiation, growth, and structure of grafted chain. Natamycin could not be attached to the untreated LDPE surface since untreated LDPE is hydrophobic. After the AA grafting, functional groups such as COOH— were coated and increased hydrophilicity which is favorable to bond with natamycin. As is shown in FIG. 4, the solvent has a significant effect on the grafting efficiency. The best grafting performance was shown in water under 4 min treatment. Water does not interact with the hydrophobic LDPE surface, and inert to the excited state of the photoinitiator (Benzophenone) while the solvent is reactive to both the grafting agent (AA) and the hydrophilic functional groups by the UV treatment. Thus, the free radical group from LDPE film may be easily abstracted and build a branch to AA without intervention. It is considered the reason water showed the highest grafting performance. Ethanol, even if it is a polar solvent like water, showed a very limited effect. The maximum amounts of natamycin grafting with ethanol was less than 36 μg/cm². Since the precoated photoinitiator (BP) is soluble in ethanol, the excited BP may interacts with the solvent and extract a hydrogen atom from the AA monomer rather than from the LDPE film. Chloroform and acetone can partially swell on the surface of polyolefin polymers (such as LDPE and HDPE), which can initiate grafting on the surface of the film more easily. Thus, these solvents are initially hypothesized as being effective for AA grafting. However, even if they are relatively mild swelling agents, they were still too strong to use on the thin LDPE cling wrap. A significant film shrinking and color change was observed after 3 min treatment. Especially, chloroform was very destructive (in observation). On the other side, Goddard and Hotchkiss (2007) reported that over-crowded reactive functional groups reduce the efficiency of grafting. This is considered another reason both acetone and chloroform were less effective for AA grafting on the film.

Organic solvents and water mixture can improve grafting reaction on film. For example, acetone can promote the methacrylic acid (MAA) solubility, and the excited triplet acetone easily abstract a secondary hydrogen atom from the grafted chain and cause branching. Thus, we performed extended the efficacy of the grafting test with various acetone and water mixtures to find the AA grafting performance. However, the mixture of acetone showed the negative effect on the grafting of AA. The percentage of grafting decreases with increasing the concentration of acetone concentration in the mixture. Since AA is a completely soluble in water, more water concentration may give the better grafting concentration in our test. Pure water is an effective solvent for AA grafting on PE films.

### Example 2.3. Natamycin Grafting

The solubility of natamycin is usually poor in water, and slightly more soluble in ethanol which is 40 ppm in pure ethanol. It was thought that the maximum solubility can be achieved in an 80% water/20% ethanol. The disclosure's results also corresponded to the result. The pH of the natamycin solution was adjusted to 5.0 using sodium phosphate buffer, to accelerate esterification reaction between a carboxylic acid (on LDPE film) and amine groups (of natamycin). Table 2 shows the various amounts of natamycin contents on the LDPE clingwrap with respect to the UV exposure time. The Orange 8 dye is supposed to be absorbed to the amine (NH) group of natamycin. The higher adsorption of amine groups was higher with the longer treatment time. The maximum amounts of natamycin was observed at 49.87 μg/cm² on 4 min treated films. Significant physical damage was observed in 5 min treated film after the 12 hr incubation with solvents. Due to the UV exposure, the surface of the thin LDPE film (cling wrap) became hydrophilic. The films easily interacted and shrunk in the warm water based solution. Thus, the film did not attain even surface area to enable it to contact with dye solution and therefore resulted in lower concentration than 3 or 4 min treated samples. Since even surface contact to food product is a very important requirement for this non-migratory active film system, such a behavior (as observed with films treated for 5 min) may not be desirable for food applications.

Table 2 shows the amounts of the grafted natamycin on treated samples (LDPE cling wrap). The quantity μg/cm² is given as the mean ± SD of triplicate measurements.

| Sample | μg/cm² |
|---|---|
| Control | 0 ± 0.00^{a} |
| 1 min | 9.25 ± 3.50^{b} |
| 2 min | 20.85 ± 2.25^{c} |
| 3 min | 41.16 ± 0.80^{e} |
| 4 min | 49.87 ± 1.50^{f} |
| 5 min | 37.00 ± 2.50^{d} |

^{a-f}For each quality parameter, means with different superscripts within a column indicate significant differences (P < 0.05).

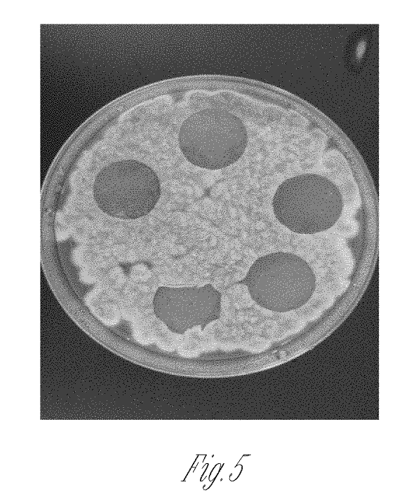

The European Parliament (European Parliament, 1995) on food additives other than color and sweeteners established that the penetration depth of natamycin into cheese should not exceed 5 mm and that the amount of natamycin on the surface should not exceed 1 mg/m² food surface (such as cantaloupe). The total grafted natamycin on the film can easily exceed the limit of 1 mg/m². However, since all non-grafted natamycin were removed by rinsing with warm distilled water, it theoretically will not migrated to food. FIG. 5 illustrates the result of diffusion cell test with the treated films, illustrating a migration assay on agar with 100 μl of P. chrysogenum inoculum. The formation of a clear zone of inhibition around the discs is not observed. It supports the theory that the antifungal agent did not migrate from the treated film. More Further studies should be followed carried out to prove with different food simulant with the use of the effectiveness FDA migration cells (ASTM, 2001).

### Example 2.4. Mechanical Properties

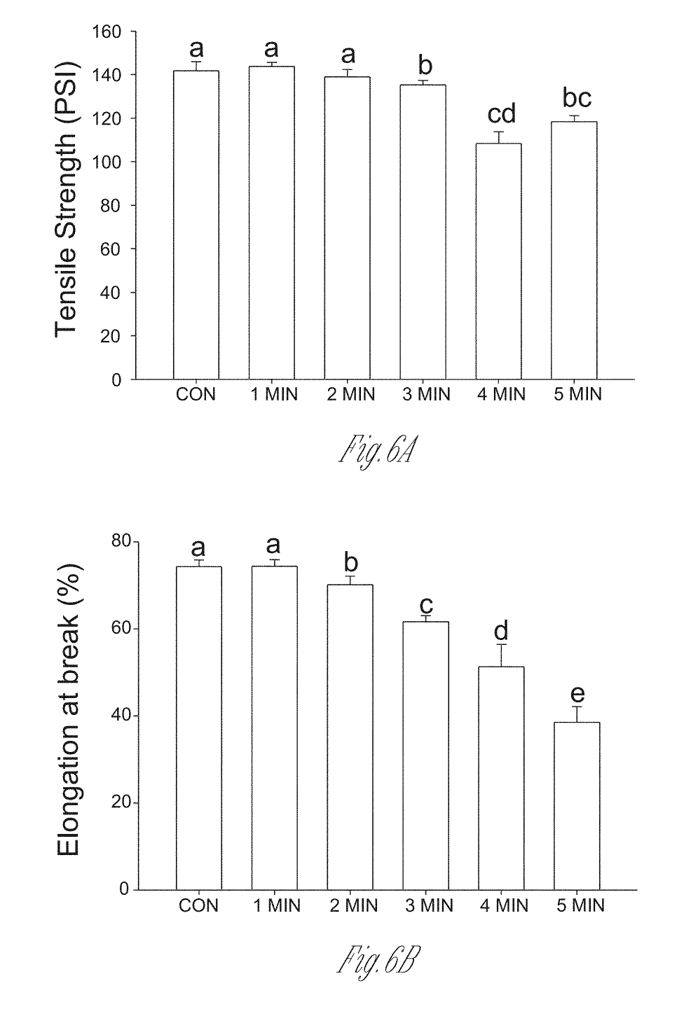

The tensile strength (TS) and % elongation at break (% E) of the LDPE cling wrap, with 1, 2, 3, 4, and 5 min UV treatments, were measured in the machine direction (FIGS. 6A-B). FIGS. 6A-B illustrates mechanical properties of the LDPE films with Natamycin under different UV treatments, with FIG. 6A illustrating tensile strength (TS) and FIG. 6B illustrating elongation at break (% E). Different superscripts indicate significant differences (P<0.05). The TS of films, which treated more than 2 min, were reduced by UV treatments. Generally, photochemical degradation causes deterioration of mechanical characteristics, cracking, and eventually complete disintegration of the polymer. Especially elongation at break of polyolefins (such as LDPE) are more sensitive to irradiation. The reduction in TS was statistically noticeable from 3 min treatment. The maximum of TS was 20% in 5 min treatment. % E was also shown to have negative effective as the UV treatment was increased. From 0-2 min, the sample did not show a clear difference to control. However, E % was reduced from 3 min treatment. In particular, the 5 min treated film showed dramatic decrease. % E was almost 50% lower than the control which is quite brittle and was not practical for use as a cling wrap. Thus, no subsequent tests for 5 min treated samples were performed in this study. 1 min treated film didn't show a statistical difference to control.

### Example 2.5. Characterization of Films Using ATR-FTIR

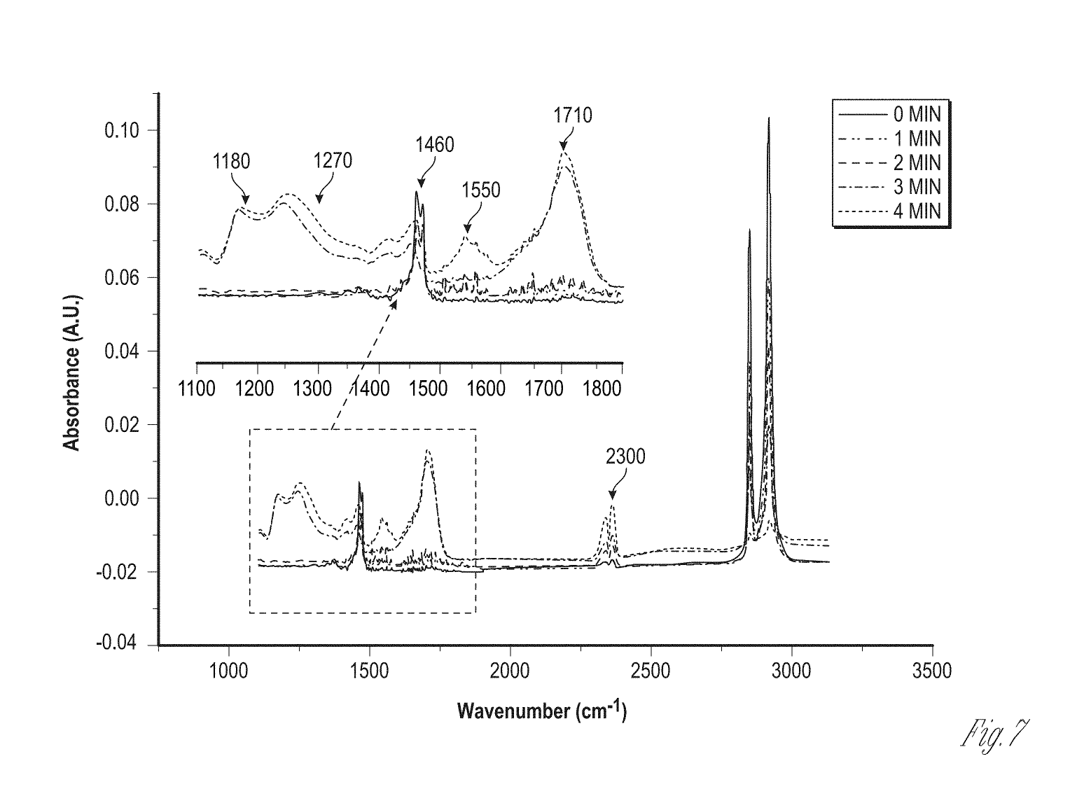

ATR-FTIR spectroscopy analysis was performed on control, and UV treated films to evaluate the surface chemistry at different treatment times (FIG. 7). A characteristic band was observed between 1550-1570 cm⁻¹, which can be attributed to the N—H bond of amine. Also, peaks between 1270-1180 cm⁻¹ showed the existence of different C—O— groups which are the typical characteristic IR absorption bands of natamycin. The absorptions of bands at about 1710 cm⁻¹ are indicative of the formation of carboxylic acid. An increasing absorbance in these bands is considered the formation of covalent bonds between the amines of natamycin and the carboxylic acid groups of AA. At about 2200-2300 cm⁻¹, double splits bands were observed, which may relate to the carbonyl group (C═O). The intensity of C═O band was increased with the highest UV exposure time. It may be due to increased degradation of the LDPE film. UV irradiation may lead to C—C bond splitting and, at the same time, to the liberation of CH₂ groups. It is considered a negative effect of UV treatment, and the intensity of the bands was dramatically increased at 4 min treated film. The result corresponded to the significant mechanical strength on the 4 min treated films (FIGS. 6A-B).

### Example 2.6. Antifungal Activity Validation

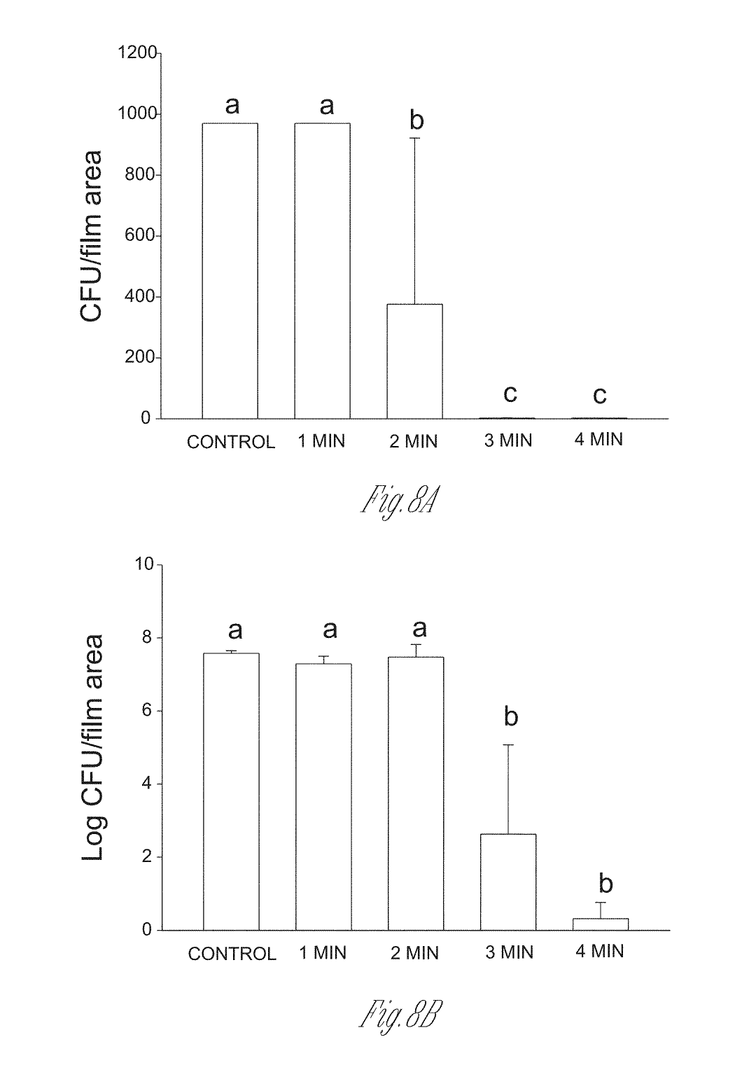

The ability of the natamycin grafted LDPE film to inactivate yeast and mold was demonstrated by incubation of control and UV-natamycin treated films. Films treated for more than 2 min with natamycin had a significant fungistatic effect on the growth inhibition of P. chrysogenum. From 918 to 970 the mold colonies were inhibited during 7 days of storage time. Enumerating mold (P. chrysogenum) on traditional agar media (DRBC) was challenging because mold grows in the form of multicellular filaments so that colonies on a petri dish rarely develop from single cells. In addition, mold easily covers the surface of the media and making enumeration difficult. This is considered the reason for such a large standard deviation which was observed on the 2 min film. The UV treated film coated with natamycin also showed a significant antifungal effect on the growth of S. cerevisiae. A 4.95 to 7.26 log reduction was observed with films treated with UV for more than 3 min (FIGS. 8A-B). FIGS. 8A-B illustrate populations of S. cerevisiae inoculated on DRBC media and overlaid with active antimicrobial film, with the area covered by the film enumerated for populations of the yeast and mold, with 8A illustrating P. chrysogenum and 8B illustrating S. cerevisiae. Different superscripts indicate significant differences (P<0.05).

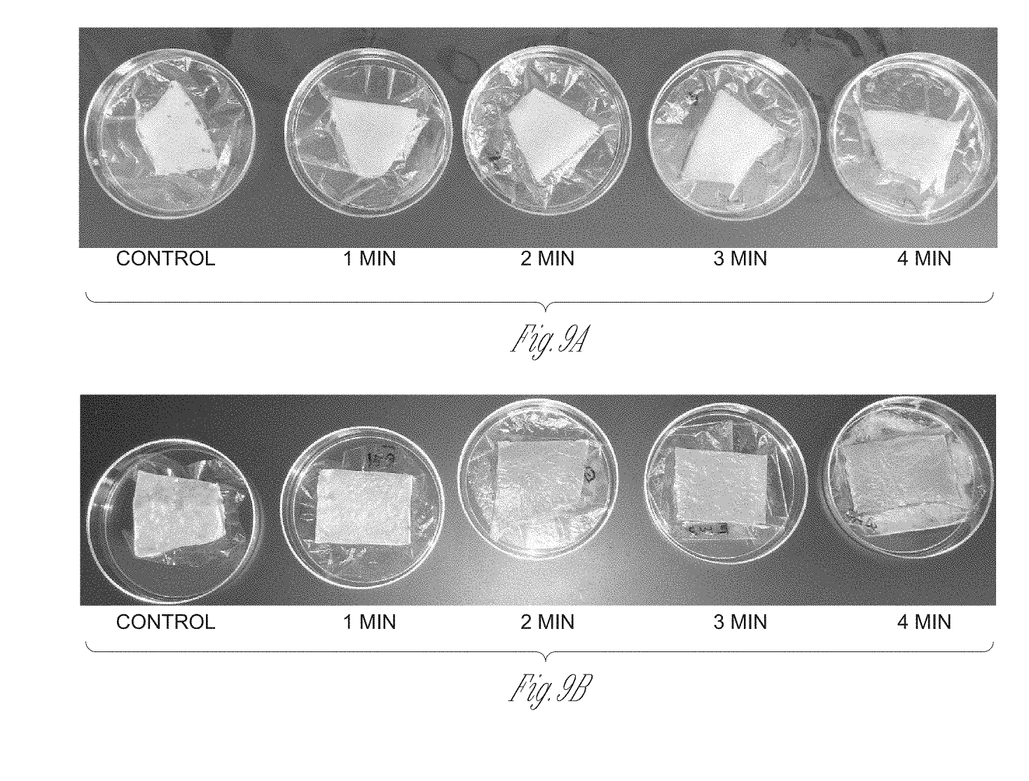

The results of the experiments on the model food systems were recorded on a qualitative basis. The resulting cling wrap antifungal films can be beneficial in enhancing the safety and quality of food such as fresh produce (e.g., whole and half-cut Melons/Cantaloupes or sliced cheeses) that do not receive terminal pasteurization treatment prior to consumption. Thus, cantaloupe was selected, as one of the most common fresh produce, and the inoculated cantaloupe piece overlaid with the films were examined on a periodic basis to determine the growth of the mold. Photographs were taken on a periodic basis to record observations (FIGS. 9A-B). FIGS. 9A-B illustrate suppression of P. chrysogenum and S. cerevisiae by UV-natamycin films on fresh cut cantaloupes, with FIG. 9A illustrating P. chrysogenum and FIG. 9B illustrating S. cerevisiae. The improved effectiveness (against both mold and yeast) was observed on this qualitative observation with cantaloupe. After the 14 day storage, no visual P. chrysogenum growth was observed in all treated films (1-4 min). A clear visual inhibition of S. cerevisiae was also observed with 2 min or longer treatment. Overall test results demonstrated antifungal function against P. chrysogenum and S. cerevisiae. The slightly more improved effect was observed on Cantaloupe instead of DRBC agar (with both mold and yeast). Since the treated surface is hydrophilic and inhibition was caused by tight contact between film and medium, the moisture content of food may be a significant factor affecting the film efficacy. Further studies are recommended to define the interaction between natamycin and moisture content of the film and its impact on efficacy.

### Example 2.7. Surface Morphology Analysis

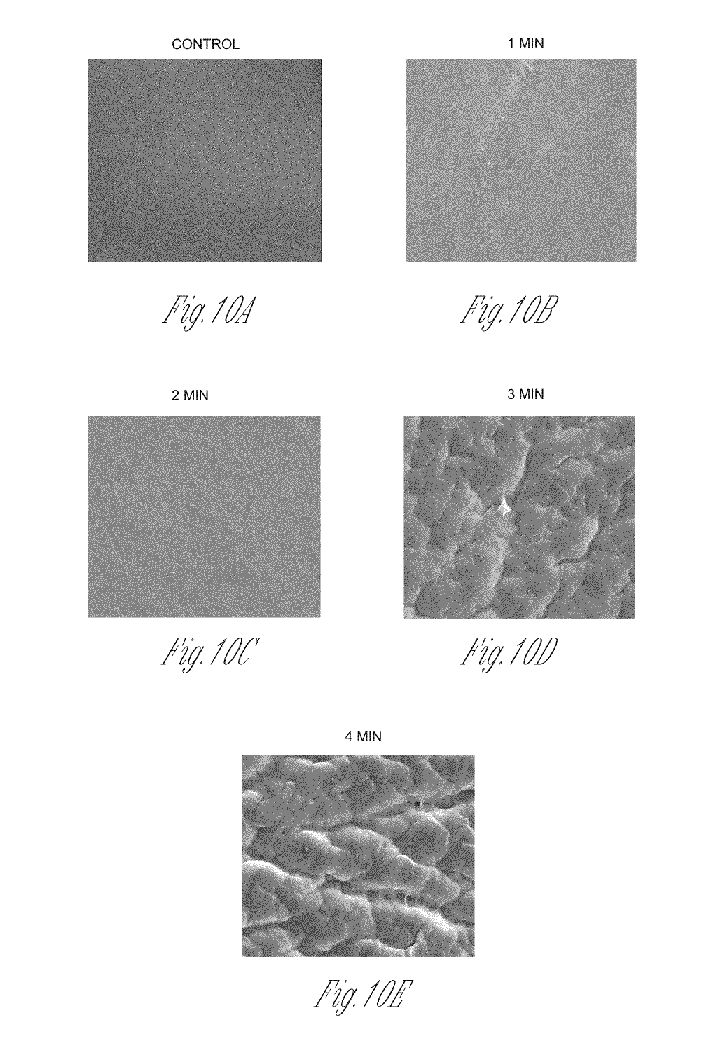

The surface morphology characteristics of control and UV-natamycin treated films are shown in FIGS. 10A-E. FIGS. 10A-E illustrate SEM images of the Natamycin coated films under different UV treatment. The control LDPE cling wrap had a smooth and continuous surface morphology. The raw film was substituted with growing aggregates after grafting. The evolution of the film surface has been proven that the presence of aggregates that started forming during a grafting reaction between the induced carboxyl groups and the enlarged AA chains. As the treatment time is increased, an increased roughness was observed for the grafted surface. The intensity of aggregation on films significantly increased in 3 and 4 min treated film.

Using the relatively simple and economical method, high levels of natamycin grafting were obtained after a short treatment time. Both gravimetric analysis and dye assay confirmed the increasing quantity of natamycin grafting on the films as UV exposure on the film was increased up to 4 min. Results indicated that the natamycin grafted LDPE cling wrap demonstrated antifungal function against P. chrysogenum and S. cerevisiae through quantitative way (4.95-7.26 log reduction in S. cerevisiae) and (918-970 spores reduction in P. chrysogenum) qualitative observation with fresh cut cantaloupes. Thus, the UV treated film containing natamycin could have potential to be used in the prevention and control of fungal contamination on fresh produces. In addition, due to the wide spread of use of natamycin to control mold growth on food products, emerged natamycin resistant fungal species have been reported such as Penicillium echinulatum and Clodosporium herbarum. Combined use with other preventive measures in a huddle concept to control such natamycin tolerable species need to be studied in the future.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Additional Embodiments

The following exemplary embodiments are provided, the numbering of which is not to be construed as designating levels of importance:

Embodiment 1 provides an antifungal-grafted polyolefin comprising an antifungal-grafted repeating unit having the structure:

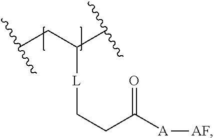

wherein
at each occurrence, -A- is chosen from —O— and —NH—,
at each occurrence, -AF is an independently selected grafted antifungal compound,
at each occurrence, -L- is independently chosen from a bond and the structure:

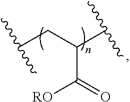

or a salt thereof,
wherein at each occurrence —R is independently chosen from —H and -AF, and at each occurrence n is independently about 1 to about 100,000.

Embodiment 2 provides the antifungal-grafted polyolefin of Embodiment 1, wherein -A- is —O—.

Embodiment 3 provides the antifungal-grafted polyolefin of Embodiment 2, wherein -AF is an antifungal compound grafted to the polyolefin via an esterification reaction of an —OH group on an ungrafted antifungal compound and a —C(O)OH group on the polyolefin.

Embodiment 4 provides the antifungal-grafted polyolefin of any one of Embodiments 1-3, wherein -A- is —NH—.

Embodiment 5 provides the antifungal-grafted polyolefin of Embodiment 4, wherein -AF is an antifungal compound grafted to the polyolefin via an amidization reaction of an —NH₂ group on an ungrafted antifungal compound and a —C(O)OH group on the polyolefin.

Embodiment 6 provides the antifungal-grafted polyolefin of any one of Embodiments 1-5, wherein the polyolefin is a homopolymer or a copolymer.

Embodiment 7 provides the antifungal-grafted polyolefin of any one of Embodiments 1-6, wherein the polyolefin further comprises another repeating unit, the other repeating unit in a block or random arrangement in the polyolefin with the antifungal-grafted repeating unit, the other repeating unit being free of the grafted antifungal compound and having the structure:

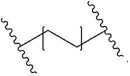

Embodiment 8 provides the antifungal-grafted polyolefin of Embodiment 7, wherein a molar ratio of the antifungal-grafted repeating unit to the other repeating unit is about 0.001:99.999 to about 99.999:0.001.

Embodiment 9 provides the antifungal-grafted polyolefin of any one of Embodiments 1-8, wherein the polyolefin is a polymer of one or more compounds that are each independently a substituted or unsubstituted (C₂-C₂₀)hydrocarbon comprising at least one carbon-carbon unsaturated nonaromatic bond.

Embodiment 10 provides the antifungal-grafted polyolefin of any one of Embodiments 1-9, wherein the polyolefin is ultra high molecular weight polyethylene (UHMWPE), ultra low molecular weight polyethylene (ULMWPE), high molecular weight polyethylene (HMWPE), high density polyethylene (HDPE), high density cross-linked polyethylene (HDXLPE), cross-linked polyethylene (PEX or XLPE), medium density polyethylene (MDPE), low density polyethylene (LDPE), linear low density polyethylene (LLDPE) and very low density polyethylene (VLDPE).

Embodiment 11 provides the antifungal-grafted polyolefin of any one of Embodiments 1-10, wherein the polyolefin is low density polyethylene (LDPE).

Embodiment 12 provides the antifungal-grafted polyolefin of any one of Embodiments 1-11, wherein the grafted antifungal compound is grafted amphotericin B, candicidin, filipin III, hamycin, natamycin, nystatin, rimocidin, efinaconazole, fluconazole, isavuconazole, posaconazole, ravuconazole, voriconazole, anidulafungin, ciclopirox, flucytosine, or grafted undecylenic acid.

Embodiment 13 provides the antifungal-grafted polyolefin of any one of Embodiments 1-12, wherein the grafted antifungal compound is grafted natamycin.

Embodiment 14 provides the antifungal-grafted polyolefin of Embodiment 13, wherein, before grafting, the natamycin has the structure:

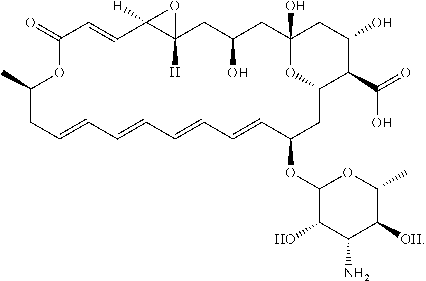 [SMILES: C[C@@H]1C/C=C/C=C/C=C/C=C/[C@H](OC2O[C@H](C)[C@@H](O)[C@H](N)[C@@H]2O)C[C@@H]2O[C@](O)(C[C@@H](O)C[C@H]3O[C@@H]3/C=C/C(=O)O1)C[C@H](O)[C@H]2C(=O)O]

Embodiment 15 provides the antifungal-grafted polyolefin of any one of Embodiments 1-14, wherein a film comprises the polyolefin.

Embodiment 16 provides the antifungal-grafted polyolefin of Embodiment 15, wherein the film is food wrap film.

Embodiment 17 provides the antifungal-grafted polyolefin of any one of Embodiments 15-16, wherein the film is about 0.01 microns to about 1 mm in thickness.

Embodiment 18 provides the antifungal-grafted polyolefin of any one of Embodiments 15-17, wherein the film is about 1 micron to about 30 microns in thickness.

Embodiment 19 provides the antifungal-grafted polyolefin of any one of Embodiments 15-18, wherein about 1 wt % to about 100 wt % of the film is the polyolefin.

Embodiment 20 provides the antifungal-grafted polyolefin of any one of Embodiments 15-19, wherein about 80 wt % to about 100 wt % of the film is the polyolefin.

Embodiment 21 provides the antifungal-grafted polyolefin of any one of Embodiments 1-20, wherein the grafted antifungal has a concentration on the polyolefin of about 0.001 microgram/cm² to about 100,000 microgram/cm².

Embodiment 22 provides the antifungal-grafted polyolefin of any one of Embodiments 1-21, wherein the grafted antifungal has a concentration on the polyolefin of about 1 microgram/cm² to about 1000 microgram/cm².

Embodiment 23 provides a method of using the antifungal-grafted polyolefin of any one of Embodiments 1-22, comprising placing the antifungal-grafted polyolefin in contact with a food item or a living human or animal body.

Embodiment 24 provides a medical device or medical implement comprising the antifungal-grafted polyolefin of any one of Embodiments 1-23.

Embodiment 25 provides a packaged food item comprising the antifungal-grafted polyolefin of any one of Embodiments 1-23.

Embodiment 26 provides the packaged food item of Embodiment 25, wherein the packaged food item comprises a food wrap film comprising the antifungal-grafted polyolefin. Embodiment 27 provides a method of forming the antifungal-grafted polyolefin of any one of Embodiments 1-23, the method comprising:

contacting a polyolefin with a free radical initiator and acrylic acid or a salt thereof to form an acrylic acid-grafted polyolefin; and

contacting the acrylic-acid-grafted polyolefin with an antifungal compound, to form the antifungal-grafted polyolefin of any one of Embodiments 1-23.

Embodiment 28 provides an antifungal film comprising:

a polyolefin film comprising an antifungal-grafted repeating unit having the structure:

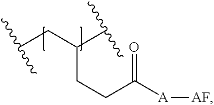

wherein
at each occurrence, -AF is a grafted natamycin, and
the grafted antifungal has a concentration on the antifungal film of about 1 microgram/cm² to about 1000 microgram/cm².

Embodiment 29 provides the antifungal-grafted polyolefin, method, medical device, medical implement, a packaged food item, or film of any one or any combination of Embodiments 1-28 optionally configured such that all elements or options recited are available to use or select from.

What is claimed is:

1. An antifungal-grafted polyolefin comprising an antifungal-grafted repeating unit having the structure: 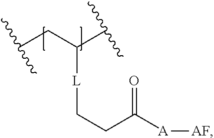
wherein
at each occurrence, -A- is chosen from —O— and —NH—,
at each occurrence, -AF is an independently selected grafted antifungal compound,
at each occurrence, -L- is independently chosen from a bond and the structure: 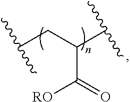
or a salt thereof,
wherein at each occurrence —R is independently chosen from —H and -AF, and at each occurrence n is independently about 1 to about 100,000.

2. The antifungal-grafted polyolefin of claim 1, wherein -A- is —O—.

3. The antifungal-grafted polyolefin of claim 2, wherein -AF is an antifungal compound grafted to the polyolefin via an esterification reaction of an —OH group on an ungrafted antifungal compound and a —C(O)OH group on the polyolefin.

4. The antifungal-grafted polyolefin of claim 1, wherein -A- is —NH—.

5. The antifungal-grafted polyolefin of claim 1, wherein the polyolefin is a homopolymer or a copolymer.

6. The antifungal-grafted polyolefin of claim 1, wherein the polyolefin further comprises another repeating unit, the other repeating unit in a block or random arrangement in the polyolefin with the antifungal-grafted repeating unit, the other repeating unit being free of the grafted antifungal compound and having the structure:

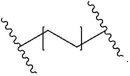

7. The antifungal-grafted polyolefin of claim 1, wherein the polyolefin is a polymer of one or more compounds that are each independently a substituted or unsubstituted (C₂-C₂₀)hydrocarbon comprising at least one carbon-carbon unsaturated nonaromatic bond.

8. The antifungal-grafted polyolefin of claim 1, wherein the polyolefin is ultra high molecular weight polyethylene (UHMWPE), ultra low molecular weight polyethylene (ULMWPE), high molecular weight polyethylene (HMWPE), high density polyethylene (HDPE), high density cross-linked polyethylene (HDXLPE), cross-linked polyethylene (PEX or XLPE), medium density polyethylene (MDPE), low density polyethylene (LDPE), linear low density polyethylene (LLDPE) and very low density polyethylene (VLDPE).

9. The antifungal-grafted polyolefin of claim 1, wherein the polyolefin is low density polyethylene (LDPE).

10. The antifungal-grafted polyolefin of claim 1, wherein the grafted antifungal compound is grafted amphotericin B, candicidin, filipin III, hamycin, natamycin, nystatin, rimocidin, efinaconazole, fluconazole, isavuconazole, posaconazole, ravuconazole, voriconazole, anidulafungin, ciclopirox, flucytosine, or grafted undecylenic acid.

11. The antifungal-grafted polyolefin of claim 1, wherein the grafted antifungal compound is grafted natamycin.

12. The antifungal-grafted polyolefin of claim 11, wherein, before grafting, the natamycin has the structure:

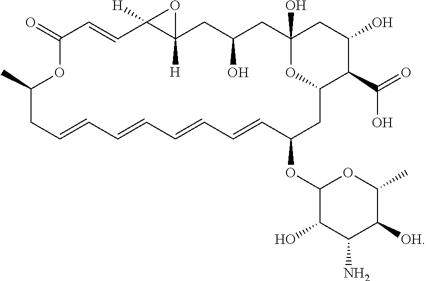 [SMILES: C[C@@H]1C/C=C/C=C/C=C/C=C/[C@H](OC2O[C@H](C)[C@@H](O)[C@H](N)[C@@H]2O)C[C@@H]2O[C@](O)(C[C@@H](O)C[C@H]3O[C@@H]3/C=C/C(=O)O1)C[C@H](O)[C@H]2C(=O)O]

13. The antifungal-grafted polyolefin of claim 1, wherein a film comprises the polyolefin.

14. The antifungal-grafted polyolefin of claim 1, wherein the grafted antifungal has a concentration on the polyolefin of about 0.001 microgram/cm² to about 100,000 microgram/cm².

15. The antifungal-grafted polyolefin of claim 1, wherein the grafted antifungal has a concentration on the polyolefin of about 1 microgram/cm² to about 1000 microgram/cm².

16. A method of using the antifungal-grafted polyolefin of claim 1, comprising placing the antifungal-grafted polyolefin in contact with a food item or a living human or animal body.

17. A medical device or medical implement comprising the antifungal-grafted polyolefin of claim 1.

18. A packaged food item comprising the antifungal-grafted polyolefin of claim 1.

19. A method of forming the antifungal-grafted polyolefin of claim 1, the method comprising:
contacting a polyolefin with a free radical initiator and acrylic acid or a salt thereof to form an acrylic acid-grafted polyolefin; and
contacting the acrylic-acid-grafted polyolefin with an antifungal compound, to form the antifungal-grafted polyolefin of claim 1.

20. An antifungal film comprising:
a polyolefin film comprising an antifungal-grafted repeating unit having the structure: 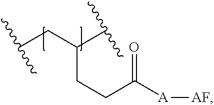
wherein
at each occurrence, -AF is a grafted natamycin, and
the grafted antifungal has a concentration on the antifungal film of about 1 microgram/cm² to about 1000 microgram/cm².
